(19) United States
(12) Patent Application Publication
(10) Pub. No.: US 2015/0125534 A1
(43) Pub. Date: May 7, 2015

(54) IMATINIB SOLID DOSAGE FORMS RECONSTITUTED JUST BEFORE USE

(71) Applicant: Mehmet Nevzat Pisak, Istanbul (TR)

(72) Inventor: Mehmet Nevzat Pisak, Istanbul (TR)

(73) Assignee: Imuneks Farma Ilac Sanayi Ve Ticaret A.S., Instanbul (TR)

(21) Appl. No.: 14/359,905

(22) PCT Filed: Nov. 30, 2011
(86) PCT No.: PCT/TR2011/000268

§ 371 Date: Jan. 16, 2015

(30) Foreign Application Priority Data
Nov. 24, 2011 (TR) 2011/11621

(51) Int. Cl. A61K 9/50; A61K 31/10; A61K 31/506

(52) U.S. Cl. CPC A61K 9/5005; A61K 31/506; A61K 9/5089; A61K 31/10
USPC 424/490; 514/252.18; 427/2.21

(57) ABSTRACT

The present invention relates to the pharmaceutical formulations comprising imatinib in solid dosage form reconstituted with a diluent just before use; preparation processes and use thereof.

### FIELD OF THE INVENTION

The present invention relates to the pharmaceutical formulations comprising imatinib in solid dosage form reconstituted with a diluent just before use; preparation processes and use thereof.

### BACKGROUND OF THE INVENTION

The present invention relates to the pharmaceutical formulations comprising imatinib in solid dosage form reconstituted with a diluent just before use.

Pharmaceutical formulations of the invention in solid dosage form reconstituted with a diluent just before use comprise imatinib in an amount from 50 mg to 800 mg, preferably in an amount from 100 mg to 600 mg, more preferably in an amount of 400 mg as unit dose. Pharmaceutical formulations of the invention comprise imatinib in an amount up to 23% by weight based on the total weight of the pharmaceutical dosage form. Pharmaceutical formulations of the invention comprise imatinib in the mesylate salt form. Imatinib mesylate of the pharmaceutical formulations of the invention is in the α crystal form.

Imatinib is a tyrosine kinase inhibitor; highly specific for BCR-ABL, the enzyme associated with chronic myelogenous leukemia and acute lymphoblastic leukemia. Imatinib is also shown to inhibit the KIT and PDGF receptors. Imatinib has the chemical name of 4-[(4-methyl-1-piperazinyl)methyl]-N-[4-methyl-3-[[4-(3-pyridinyl)-2-pyrimidinyl]amino]phenyl] benzamide. Imatinib has the chemical structure of Formula 1.

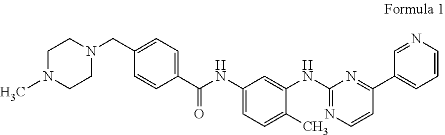 [SMILES: Cc1ccc(NC(=O)c2ccc(CN3CCN(C)CC3)cc2)cc1Nc1nccc(-c2cccnc2)n1]

Imatinib is currently available in the market in capsule and film tablet dosage forms with the brand name Glivec in Turkey and Europe; and in film tablet dosage form with the brand name Gleevec in United States of America. Common imatinib doses are between 50 mg and 800 mg in orally administered tablet or capsule form for adult patients.

Imatinib is used in the treatment of certain types of cancer, such as chronic myelogenous leukemia and acute lymphoblastic leukemia. Cancer is known to be a disease that reduces the quality of life of the patients due to the course of the disease, symptoms and complications; and also due to hard treatment methods and adverse effects resulting from the treatment. Therefore, increasing patient compliance to the treatment in both physical and psychological ways is crucial for the success of the treatment. Physical and psychological state of the patient, patient's perspective of the disease and the treatment are determining factors within this process. For instance, the patient may have difficulty in swallowing due to psychological causes, age, symptoms of the disease or adverse effects resulting from the treatment; and may not be able to continue the treatment properly. Such a non-compliance between the patient and the treatment significantly affects the success of the treatment of which each stage has vital importance.

The present invention encapsulates pharmaceutical formulations that increase patient compliance to the treatment, that can be easily used and that do not cause difficulty in swallowing in contrary to the medicines available in the market in capsule and tablet dosage forms. Pharmaceutical formulations of the invention are stable pharmaceutical formulations in solid dosage form reconstituted with a diluent just before use. Pharmaceutical formulations of the invention comprise imatinib in the mesylate salt form. Imatinib mesylate of the pharmaceutical formulations of the invention is in the α crystal form.

Any information relating to the pharmaceutical formulations of the invention in solid dosage form reconstituted with a diluent just before use has not been disclosed in the prior art. Said prior art documents are as follows:

EP1501485 B1 relates to the tablets comprising a pharmacologically effective amount of imatinib or a pharmaceutically acceptable salt thereof in an amount from 30% to 80% in weight of the active moiety based on the total weight of the tablet.

TR2008/02061 A2 relates to the tablets comprising imatinib or a pharmaceutically acceptable salt thereof, a hydrate thereof or a salt of hydrate thereof in an amount above 80% in weight of the active moiety based on the total weight of the tablet, together with pharmaceutically acceptable excipients.

EP1762230 B1 relates to a process for the preparation of a film-coated tablet characterised in that, prior to pressing of the starting materials, at least imatinib mesylate is dry-granulated; the tablet cores containing imatinib mesylate in an amount of from 25 wt. % to 80 wt. %, based on the total weight of the tablet cores are obtained; and the resulting tablet cores so prepared are coated with a film coating.

EP2068835 A2 relates to the solid solutions comprising imatinib and a solid solvent. Solid solution of said invention is then granulated, granules so-obtained are mixed with at least one excipient to obtain final mixture, said final mixture is tabletted and optionally coated.

EP2081556 A1 relates to the pharmaceutical formulations comprising an initial polymorphic form of imatinib mesylate, wherein less than 10% of the polymorphic form of imatinib mesylate is converted to form α or form β after storage at 40° C. at 75% relative humidity for 1 month, wherein the initial polymorphic form of imatinib mesylate is imatinib mesylate form V or imatinib mesylate form X.

CN101401797 A, CN101401795 A and CN101401794 A relate to effervescent tablet, orally disintegrating tablet and chewable tablet formulations comprising imatinib mesylate.

Above-mentioned prior art documents only relate to tablet forms of imatinib. Imatinib is currently marketed worldwide in only tablet and capsule forms. Accordingly, any information relating to the pharmaceutical formulations of the invention in solid dosage form reconstituted with a diluent just before use has not been disclosed in said prior art documents.

EP2120877 A2 relates to the solid dispersions comprising imatinib mesylate in an amorphous form and a carrier, wherein said carrier is a cellulose derivative. EP2000139 A1 relates to the pharmaceutical formulations comprising a cyclodextrin complex that stabilizes amorphous imatinib mesylate, optionally together with at least one carrier.

Above-mentioned prior art documents relate to the use of imatinib mesylate in amorphous form. However, pharmaceutical formulations of the invention comprise imatinib mesylate in the α crystal form.

EP1888040 B1 relates to the sustained release pharmaceutical formulations comprising melt granules of at least 400 mg of imatinib mesylate and a release retardant. EP2086516 A1 relates to the sustained release pharmaceutical formulations comprising a melt-processed mixture of imatinib or a salt thereof, at least one polymeric binder and at least one non-ionic surfactant.

Above-mentioned prior art documents relate to the formulations of imatinib of which release characteristics are changed by melt-processing. However, release of pharmaceutical formulations of the invention is extended by enteric coating of imatinib granules obtained by wet granulation.

EP2268265 A2 relates to the nanoparticulate formulations comprising an imatinib compound and an enteric matrix or enteric coating or a combination thereof; whereby at least 80% of the imatinib compound is released in the small intestine.

Above-mentioned prior art document relates to the nanoparticulate tablet formulations of imatinib produced by a totally different process; and having different particle size and extended release characteristics. However, pharmaceutical formulations of the invention are not in tablet form and comprise imatinib in conventional particle size.

As a result, the present invention discloses novel and stable pharmaceutical formulations comprising imatinib in solid dosage form reconstituted with a diluent just before use.

### SUMMARY OF THE INVENTION

The present invention relates to a pharmaceutical powder formulation wherein the powder formulation comprises enteric coated imatinib granules and imatinib is present in an amount up to 23% by weight based on the total weight of the powder formulation.

### DETAILED DESCRIPTION OF THE INVENTION

The present invention encompasses stable pharmaceutical formulations comprising imatinib in solid dosage form reconstituted with a diluent just before use.

The present invention entails a powder formulation that increases patient compliance to the treatment, that can be easily used and that does not cause difficulty in swallowing compared to the other dosage forms of imatinib available in the market as capsule and tablet dosage forms. Pharmaceutical formulations of the invention are stable pharmaceutical formulations in solid dosage form reconstituted with a diluent just before use. Because the pharmaceutical formulations of the invention are in solid dosage form reconstituted with a diluent just before use, the patients will not have difficulty in swallowing. Furthermore, the patients will be able to use this formulation as a drink and this will affect the psychological state of the patient positively. Hence, the patients will have higher compliance to the treatment and therefore, the treatment's success rate will increase.

However, gastric irritation caused by medications comprising imatinib is a common serious adverse effect. Taking a medication comprising imatinib directly may cause larynx, pharinx and esophagus irritation in addition to gastric irritation. Such adverse effects are barriers that decrease patient compliance to the treatment. Pharmaceutical formulations of the invention comprise enteric coated imatinib granules in powder form in order to prevent a possible irritation. Taking the pharmaceutical formulations of the invention by adding into a diluent also reduces the irritation.

The reason for formulating the pharmaceutical formulations comprising imatinib in solid dosage form reconstituted with a diluent just before use is to increase the compliance of the patient to the treatment, but developing solubility and stability characteristics of said pharmaceutical formulations for providing the treatment success was needed. Solubility is dependent on critical parameters such as temperature, presence of electrolytes (salting-out effect), state of crystallinity (amorphous, crystal form), nature of crystals and complexation. Stability is dependent on critical parameters such as chemical composition, particle size and environmental factors (air, light, temperature . . . ).

Pharmaceutical formulations of the invention comprise imatinib in the mesylate salt form; because, while imatinib free base is practically insoluble in water, mesylate salt of imatinib displays good solubility and stability.

Imatinib mesylate of the pharmaceutical formulations of the invention is in the α crystal form. Form α and Form β polymorphs show similar aqueous solubility characteristics. However, Form α is described to be hygroscopic, there was a need to enable the stability by preventing imatinib mesylate of the pharmaceutical formulations of the invention in the α crystal form from absorbing moisture. Formulating the pharmaceutical formulations of the invention in powder form comprising enteric coated imatinib granules has prevented imatinib mesylate in the α crystal form from absorbing moisture. As a result, pharmaceutical powder formulations of the invention comprising enteric coated imatinib granules display good solubility and stability.

Pharmaceutical formulations of the invention are in an orally administered dosage form of powder or granule for sachet, liquid, solution, suspension, emulsion or syrup; preferably in the form of powder for suspension comprising enteric coated imatinib granules or in the form of powder for sachet comprising enteric coated imatinib granules.

Therapeutically effective amount of imatinib free base in the pharmaceutical dosage forms of the invention is an amount from 50 mg to 800 mg, preferably an amount from 100 mg to 600 mg, more preferably an amount of 400 mg as unit dose. Pharmaceutical dosage forms of the invention comprise imatinib in an amount up to 23% (<23%) by weight based on the total weight of the pharmaceutical dosage form. Said ratio is calculated based on the mesylate salt of imatinib. Pharmaceutical dosage forms of the invention may be used once or twice a day.

Oral dosage forms of the invention comprise at least one pharmaceutically acceptable excipient selected from the group comprising filler, binder, disintegrant, lubricant, antioxidant, surfactant, solubility enhancing agent, pH modulating agent, viscosity modulating agent, preservative, sweetener, flavoring agent, coloring agent and coating agent.

Pharmaceutically acceptable filler may be selected from the group comprising lactose, microcrystalline cellulose, starch, pre-gelatinized starch, calcium phosphate, calcium sulfate, calcium carbonate, mannitol, sorbitol, xylitol, sucrose, maltose, fructose, dextrose, maltodextrin, and the like.

Pharmaceutically acceptable binder may be selected from the group comprising starches, natural sugars, corn sweeteners, natural and synthetic gums, cellulose derivatives, gelatin, povidone, polyethylene glycol, waxes, sodium alginate, alcohols, water, and the like.

Pharmaceutically acceptable disintegrant may be selected from the group comprising starches, cellulose derivatives, povidone, crospovidone, clays, ion exchange resins, alginic acid, sodium alginat, and the like.

Pharmaceutically acceptable lubricant may be selected from the group comprising metallic stearates, metallic lauryl sulfates, fatty acids, fatty acid esters, fatty alcohols, paraffins, hydrogenated vegetable oils, polyethylene glycols, boric acid, sodium benzoate, sodium acetate, sodium chloride, talk, and the like.

Pharmaceutically acceptable antioxidant may be selected from the group comprising citric acid, malic acid, fumaric acid, ascorbic acid, ascorbates, ascorbyl palmitate, EDTA, butylated hydroxyanisole, butylated hydroxytoluene, lecithin, sulfuric acid salts, tocopherols, gallates, thiols, polyphenols, and the like.

Pharmaceutically acceptable surfactant may be selected from the group comprising sulfates, sulfonates, phosphates, carboxylates, primary-secondary-tertiary amines, quaternary ammonium compounds, fatty alcohols, sugar esters of fatty acids, glycerides of fatty acids, polyoxyethylene glycol alkyl ethers, polysorbates, sorbitan alkyl esters, poloxamers, and the like.

Pharmaceutically acceptable solubility enhancing agent may be selected from the group comprising cosolvents (glycerol, ethanol, sorbitol, propylene alcohol, polyethylene glycol, and the like), surfactants and complex forming agents (β-cyclodextrins, povidone, and the like).

Pharmaceutically acceptable pH modulating agent may be selected from the group comprising adipic acid, acetic acid, ascorbic acid, phosphoric acid, fumaric acid, glutamic acid, hydrochloric acid, lactic acid, malic acid, nitric acid, citric acid, sodium hydroxide, sodium chloride, succinic acid, sulfuric acid, tartaric acid, and the like.

Pharmaceutically acceptable viscosity modulating agent may be selected from the group comprising colloidal silicon dioxide, cellulose derivatives, povidone, sugar, xanthan gum, gelatin, and the like.

Pharmaceutically acceptable preservative may be selected from the group comprising parabens, phenol, chlorocresol, parahydroxy benzoic acid alkyl esters, benzoic acid and salts thereof, boric acid and salts thereof, citric acid and salts thereof, sorbic acid and salts thereof, neutral preservatives, mercurial preservatives, quaternary compounds, and the like.

Pharmaceutically acceptable sweetener may be selected from the group comprising alitame, acesulfame potassium, aspartame, D-tryptophan, dextrose, erythritol, fructose, galactose, glycerol, glycyrrhizin, glucose, isomalt, xylitol, xylose, lactitol, lactose, levulose, maltitol, maltodextrin, maltol, maltose, mannitol, corn syrup, neohesperidin dihydrochalcone, neotame, saccharin, siclamate, sorbitol, sucralose, sucrose, tagatose, taumatin, trehalose, and the like.

Pharmaceutically acceptable flavoring agent may be selected from the group comprising natural flavoring oils, anethole, acetic acid, ascorbic acid, phosphoric acid, fumaric acid, lactic acid, lemon, linalool, malic acid, menthol, eucalyptol, orange, citric acid, cinnamone, tartaric acid, thymol, vanilla, strawberry, and the like.

Pharmaceutical formulations of the invention are for the treatment of certain types of cancer, such as chronic myelogenous leukemia and acute lymphoblastic leukemia. Pharmaceutical formulations of the invention may be used once or twice a day as unit doses.

### Pharmaceutical Formulations of the Invention in the Dosage Form of Powder for Suspension or Powder for Sachet Comprising Enteric Coated Imatinib Granules

Based on the studies, it has been found that powder for suspension or powder for sachet formulations of the invention comprising imatinib (imatinib mesylate in the α crystal form) in an amount up to 23% by weight based on the total weight of powder for suspension or powder for sachet formulation comprising enteric coated imatinib granules display good solubility and stability.

Preparation process of the formulations of the invention consists of two stages. First stage comprises the steps of preparing granules comprising imatinib mesylate α crystals by wet granulation and enteric coating so-obtained granules. Second stage comprises the step of preparing powder mixture for suspension or powder mixture for sachet by adding excipients of the invention into the enteric coated imatinib mesylate granules.

First stage comprises following steps:
a) dissolving at least one binder in purified water;
b) sieving and mixing imatinib mesylate and at least one excipient; and loading the mixture into the fluid bed granulator;
c) wet granulation by spraying the solution obtained in step a) into the mixture obtained in step b);
d) drying the granules obtained in step c);
e) enteric coating the dried granules in fluid bed granulator.

Second stage comprises following steps:
f) sieving and mixing enteric coated imatinib mesylate granules and at least one excipient;
g) filling the final mixture into the bottles for suspension form or into the sachets for sachet form.

Unit dose of pharmaceutical dosage forms of the invention may be taken directly; or by adding into a diluent such as half or full glass of water, fruit juice, or syrup depending on the dosage form. For instance, powder mixture for suspension filled into the bottles is reconstituted with water to the volume (50, 100 or 150 ml), each unit dose of said suspension is taken directly; or by adding it into a diluent. Powder mixture for sachet filled into the sachets is taken by adding it into a diluent.

Pharmaceutical dosage forms prepared by the process of the invention display high solubility. Powder mixture comprising granules obtained by the preparation process of the invention display >80% solubility within first 20 minutes in the dissolution medium.

Pharmaceutical dosage forms prepared by the process of the invention have not met any stability problems during long term stability studies performed at 25±2° C. and 60±5% RH across a 0-, 3- and 6-month follow-up period; and accelerated stability studies performed at 40±2° C. and 75±5% RH across a 0-, 3- and 6-month follow-up period.

Use of pharmaceutical dosage forms prepared by the process of the invention is easier than that of conventional dosage forms. Risk of irritation caused by said dosage forms is significantly lower than that of conventional dosage forms. Consequently, said dosage forms will increase patient compliance to the treatment as well as the success of the treatment.

The extent of the invention should not be limited by the examples of the pharmaceutical formulations and preparation processes of the invention. The examples below are only given to illustrate the invention.

### EXAMPLE 1

### Powder Formulation for Suspension Comprising 400 mg/5 ml Imatinib in 50 ml Bottle

| Unit formula of powder for suspension | | |
|---|---|---|
| comprising imatinib mesylate equivalent | Amount in | Ratio in |
| to 400 mg/5 ml imatinib | bottle (g) | bottle (%) |
| Imatinib mesylate* | 4.779 | 21.240 |
| Microcrystalline cellulose | 1.016 | 4.516 |
| Povidone | 0.637 | 2.831 |
| Hypromellose phthalate | 6.212 | 27.609 |
| Castor oil | 0.646 | 2.871 |
| Colloidal silicondioxide | 0.050 | 0.222 |
| Potassium sorbate | 0.100 | 0.444 |
| Xanthan gum | 0.019 | 0.084 |
| Citric acid anhydrous | 0.021 | 0.093 |
| Strawberry flavor | 0.090 | 0.400 |
| Titanium dioxide | 0.178 | 0.791 |
| Maltodextrin | 1.190 | 5.289 |
| Sugar (Fine Granule) | 7.562 | 33.609 |
| Total | 22.500 | 100.000 |

*Imatinib mesylate equivalent to 4.000 g and 17.778% of imatinib

### EXAMPLE 2

### Powder Formulation for Suspension Comprising 400 mg/5 ml Imatinib in 100 ml Bottle

| Unit formula of powder for suspension | | |
|---|---|---|
| comprising imatinib mesylate equivalent | Amount in | Ratio in |
| to 400 mg/5 ml imatinib | bottle (g) | bottle (%) |
| Imatinib mesylate* | 9.558 | 21.240 |
| Microcrystalline cellulose | 2.032 | 4.516 |
| Povidone | 1.274 | 2.831 |
| Hypromellose phthalate | 12.424 | 27.609 |
| Castor oil | 1.292 | 2.871 |
| Colloidal silicondioxide | 0.100 | 0.222 |
| Potassium sorbate | 0.200 | 0.444 |
| Xanthan gum | 0.038 | 0.084 |
| Citric acid anhydrous | 0.042 | 0.093 |
| Strawberry flavor | 0.180 | 0.400 |
| Titanium dioxide | 0.356 | 0.791 |
| Maltodextrin | 2.380 | 5.289 |
| Sugar (Fine Granule) | 15.124 | 33.609 |
| Total | 45.000 | 100.000 |

*Imatinib mesylate equivalent to 8.000 g and 17.778% of imatinib

### EXAMPLE 3

### Powder Formulation for Suspension Comprising 400 mg/5 ml Imatinib in 150 ml Bottle

| Unit formula of powder for suspension | | |
|---|---|---|
| comprising imatinib mesylate equivalent | Amount in | Ratio in |
| to 400 mg/5 ml imatinib | bottle (g) | bottle (%) |
| Imatinib mesylate* | 14.337 | 21.240 |
| Microcrystalline cellulose | 3.048 | 4.516 |
| Povidone | 1.911 | 2.831 |
| Hypromellose phthalate | 18.636 | 27.609 |
| Castor oil | 1.938 | 2.871 |
| Colloidal silicondioxide | 0.150 | 0.222 |
| Potassium sorbate | 0.300 | 0.444 |
| Xanthan gum | 0.057 | 0.084 |
| Citric acid anhydrous | 0.063 | 0.093 |
| Strawberry flavor | 0.270 | 0.400 |
| Titanium dioxide | 0.534 | 0.791 |
| Maltodextrin | 3.570 | 5.289 |
| Sugar (Fine Granule) | 22.686 | 33.609 |
| Total | 67.500 | 100.000 |

*Imatinib mesylate equivalent to 12.000 g and 17.778% of imatinib

### EXAMPLE 4

### Powder Formulation for Sachet Comprising 400 mg Imatinib in a Sachet

| Unit formula of powder for 1 sachet comprising | Amount in | Ratio in |
|---|---|---|
| imatinib mesylate equivalent to 400 mg imatinib | sachet (g) | sachet (%) |
| Imatinib mesylate* | 0.478 | 21.244 |
| Microcrystalline cellulose | 0.102 | 4.533 |
| Povidone | 0.064 | 2.844 |
| Hypromellose phthalate | 0.621 | 27.600 |
| Castor oil | 0.064 | 2.844 |
| Colloidal silicondioxide | 0.005 | 0.222 |
| Potassium sorbate | 0.010 | 0.444 |
| Xanthan gum | 0.002 | 0.089 |
| Citric acid anhydrous | 0.002 | 0.089 |
| Strawberry flavor | 0.009 | 0.400 |
| Titanium dioxide | 0.018 | 0.800 |
| Maltodextrin | 0.119 | 5.289 |
| Sugar (Fine Granule) | 0.756 | 33.600 |
| Total | 2.250 | 100.000 |

*Imatinib mesylate equivalent to 0.400 g and 17.778% of imatinib

### EXAMPLE 5

### Preparation Process of the Powder Formulation for Suspension or Powder Formulation for Sachet Comprising 400 mg Imatinib as Unit Dose

Preparation process of the formulations of the invention consists of two stages. First stage comprises the steps of preparing granules comprising imatinib mesylate α crystals by wet granulation and enteric coating so-obtained granules. Second stage comprises the step of preparing the powder mixture for suspension or powder mixture for sachet by adding excipients of the invention into the enteric coated imatinib mesylate granules.

First stage comprises following steps:
a) dissolving a certain amount of povidone in purified water;
b) sieving imatinib mesylate, microcrystalline cellulose and remaining part of povidone with vibrating sieve; mixing them; and loading the mixture into the fluid bed granulator with vacuum;
c) wet granulation by spraying the povidone solution obtained in step a) into the mixture obtained in step b);
d) drying the granules obtained in step c) in dry granulator;
e) coating the dried granules with hypromellose phthalate, castor oil, water, ethanol and acetone in fluid bed granulator.

Second stage comprises following steps:
f) sieving enteric coated imatinib mesylate granules and colloidal silicon dioxide, potassium sorbate, xanthan gum, citric acid anhydrous, strawberry flavor, titanium dioxide, maltodextrin and sugar with fine granule with vibrating sieve; and mixing them;
g) filling the final mixture into the bottles for suspension form or into the sachets for sachet form.

1-24. (canceled)

25. A pharmaceutical powder formulation comprising granules of a tyrosine kinase inhibitor, wherein the granules of the tyrosine kinase inhibitor are coated with an enteric coating, wherein the tyrosine kinase inhibitor is present in an amount of up to 23% by weight based on the total weight of the pharmaceutical powder formulation.

26. The pharmaceutical powder formulation of claim 25, wherein the tyrosine kinase inhibitor is imatinib, or a pharmaceutically-acceptable salt thereof.

27. The pharmaceutical powder formulation of claim 26, wherein the pharmaceutically-acceptable salt is a mesylate salt.

28. The pharmaceutical powder formulation of claim 25, wherein the tyrosine kinase inhibitor is in an α-crystal form.

29. The pharmaceutical powder formulation of claim 25, wherein the pharmaceutical powder formulation is a unit dosage form, and further comprises a pharmaceutically-acceptable excipient.

30. The pharmaceutical powder formulation of claim 29, wherein the pharmaceutically-acceptable excipient is a binder.

31. The pharmaceutical powder formulation of claim 25, wherein the tyrosine kinase inhibitor is present in an amount from 17% to 23% by weight based on the total weight of the pharmaceutical powder formulation.

32. The pharmaceutical powder formulation of claim 25, wherein the tyrosine kinase inhibitor is present in an amount from 50 mg to 800 mg.

33. The pharmaceutical powder formulation of claim 25, wherein the tyrosine kinase inhibitor is present in an amount from 100 mg to 600 mg.

34. The pharmaceutical powder formulation of claim 25, wherein the tyrosine kinase inhibitor is present in an amount of 400 mg.

35. A process for preparation of a pharmaceutical powder formulation, the process comprising:
a) dissolving a binder in purified water to provide a solution;
b) mixing a tyrosine kinase inhibitor and a first excipient to provide a mixture;
c) performing wet granulation by spraying the solution of binder in purified water into the mixture of the tyrosine kinase inhibitor and the first excipient to provide granules;
d) drying the granules obtained from c) to provide dried granules; and
e) applying an enteric coating to the dried granules to provide coated granules.

36. The process of claim 35, wherein the coated granules are in powder form.

37. The process of claim 35, wherein the coated granules are in sachet form.

38. The process of claim 35, further comprising reconstitution of the powder to a volume to provide a unit dose.

39. The process of claim 35, wherein the tyrosine kinase inhibitor is imatinib, or a pharmaceutically-acceptable salt thereof.

40. The process of claim 39, wherein the pharmaceutically-acceptable salt is a mesylate salt.

41. The process of claim 35, wherein the tyrosine kinase inhibitor is in an α-crystal form.

42. A process for treating cancer, the process comprising:
a) reconstituting a pharmaceutical powder formulation to a volume to provide a unit dose, wherein the pharmaceutical powder formulation comprises granules of a tyrosine kinase inhibitor; and
b) administering the unit dose to a patient.

43. The process of claim 42, wherein the tyrosine kinase inhibitor is imatinib, or a pharmaceutically-acceptable salt thereof.

44. The process of claim 43, wherein the pharmaceutically-acceptable salt is a mesylate salt.

45. The process of claim 42, wherein the tyrosine kinase inhibitor is in an α-crystal form.

46. The process of claim 42, the process further comprising adding a diluent to the unit dose.

47. The process of claim 42, wherein the tyrosine kinase inhibitor is present in an amount from 17% to 23% by weight based on the total weight of the pharmaceutical powder formulation.

48. The process of claim 42, wherein the granules of the tyrosine kinase inhibitor are coated with an enteric coating.

49. The process of claim 42, wherein the tyrosine kinase inhibitor is present in an amount from 50 mg to 800 mg.

50. The process of claim 42, wherein the tyrosine kinase inhibitor is present in an amount from 100 mg to 600 mg.

51. The process of claim 42, wherein the tyrosine kinase inhibitor is present in an amount of 400 mg.

52. The process of claim 42, wherein the unit dose is administered to the patient once a day.

53. The process of claim 42, wherein the unit dose is administered to the patient twice a day.

54. The process of claim 42, wherein the cancer is chronic myelogenous leukemia.

55. The process of claim 42, wherein the cancer is acute lymphoblastic leukemia.
